(19) United States
(12) Patent Application Publication
(10) Pub. No.: US 2007/0259050 A1
(43) Pub. Date: Nov. 8, 2007

(54) Combined Cosmetic or Therapeutic Preparation

(75) Inventors: Gabriele Blume, Steinau a.d. Strasse (DE); Dirk Teichmuller, Linsengericht (DE)

Correspondence Address: MICHAEL L. DUNN, SIMPSON & SIMPSON, PLLC, 5555 MAIN STREET, WILLIAMSVILLE, NY, 14221, US

(73) Assignee: Rovi GmbH & Co. Kosmetishe Rotstoffe Kg, Schluchtern (DE)

(21) Appl. No.: 10/579,121

(22) PCT Filed: Nov. 4, 2004
(86) PCT No.: PCT/EP04/52792

§ 371 Date: Mar. 12, 2007

(30) Foreign Application Priority Data
Nov. 11, 2003 (DE) 10352602.1

(51) Int. Cl. A61K 31/34; A61K 31/52; A61K 31/715; A61K 31/727; A61K 35/62; A61K 36/16; A61K 47/44; A61P 17/00; A61P 9/14

(52) U.S. Cl. 424/537; 424/752; 514/17; 514/263.1; 514/456; 514/457; 514/461; 514/56; 514/772

(57) ABSTRACT

A combined cosmetic or therapeutic preparation having a carrier system comprising membrane-forming lipids and at least two active ingredients which are selected from at least two of the groups (a) anti-coagulants, (b) vasoprotective agents and (c) microcirculation-promoting substances. The invention also includes the method for making the preparation from its ingredients.

### BACKGROUND OF THE INVENTION

The present invention concerns a combined cosmetic or therapeutic preparation with a carrier system comprising membrane-forming lipids and at least two active ingredients.

The vessel walls of the blood circulation system are continuously subjected to a high loading. High pressures are built up in the arterial system due to the active pumping activity of the heart. The venous system readily suffers from pooling of the transported blood, in particular in the extremities, as transport of the blood takes place largely against the force of gravity. In the capillary system (also referred to as the end flow path) a considerable resistance is opposed to the flow of blood by virtue of the extremely small diameters of the capillaries. The movement of the blood in the end flow path is also referred to as microcirculation, in which case the blood flow speed drops to about 0.5 mm/sec.

Essentially, blood vessels comprise three layers. The innermost layer forms a single-layer pavement epithelium (endothelium) with superposed basal membrane, wherein “single-layer” signifies that generally this actually involves only a single layer of cells. The middle layer substantially comprises smooth musculature and elastic fibres. While the musculature is responsible for constricting or dilating the vessels, the fibres provide for the elasticity thereof. The outermost layer is also elastic and substantially comprises connective tissue. A distinguishing feature for arteries and veins is that the muscle layer in arteries is of a markedly thicker nature. The muscle layer of the veins is accordingly thinner, it can in part also be entirely omitted and is to be found in particular in the large veins.

Impairment of the stability of the vessel walls generally leads to a reduction in the vessel wall sealing integrity, and the consequence of that can be a series of various symptoms. The diffuse, mostly painless accumulation of serous liquid which has escaped from the vessel system, in the tissue gaps of different tissues, is referred to as tissue dropsy or edema. Edema which is low in protein occurs for example due to an increase in the intravasal hydrostatic pressure or due to a reduction in the intravasal colloidosmotic pressure. Edema which is rich in protein is the consequence of an increase in vessel wall permeability. The increase in vessel wall permeability is frequently involved with dilation and occurs inter alia in the context of inflammations. In the case of a local inflammation reaction, transudation of plasma and also transmigration of blood cells occurs.

A hematoma or bruise is a traumatically induced accumulation of blood outside the vessels (blood extravasation). The hematoma can be formed in the tissue or in a previously formed cavity where it gradually coagulates and in part becomes streaky like connective tissue. A known hematoma at the surface of the body with the typical colour changes is the subcutaneous hematoma, a bruise in the subcutaneous tissue. As a consequence of hemoglobin breakdown that involves the characteristic colouring of the initially blue-red blood stains to yellowish-greenish blood bumps. Besides the hematomas which occur due to mechanical damage (in particular blunt trauma by knocking against hard objects), bruises can also occur due to an overloading of the capillary system as a consequence of for example stress. Such a hematoma can be formed for example in the lower eyelid and lead to dark shadows which in colloquial speech are referred to as “dark rims of the eyes” or “rings under the eyes”.

In regard to the production of those dark shadows, blood does not necessarily have to escape from the vessels. The very slow flow of blood and the simultaneous depletion of oxygen in the blood, accompanied by the darker colouring which is characteristic of venous blood, can also be the cause of such shadows. A similar situation arises in regard to the blood vessels which are coloured from bright red to dark violet and which are frequently to be observed for example on the legs, being referred to in colloquial speech as “witches' broomsticks” or “broom twigs”.

The above-described symptoms are frequently combined together under the term venous insufficiency. Venous insufficiency is often accompanied by pain, and a feeling of stress and heaviness. Swellings can also be pronounced in the legs, in particular in the evening and on hot days. The retention of water also means that there is a reduced supply of oxygen to the tissue. Hereinafter the complex of symptoms which, by virtue of increased vessel wall permeability, is accompanied by the formation of hematomas and edemas in the tissue, is referred to as vessel wall insufficiency and therefore embraces both the venous system and also the arterial system.

Even if it is not yet entirely clear from the scientific point of view why vessel walls such as for example in the lower eyelid become weak, some active ingredients are known from the state of the art for the prophylactic or therapeutic treatment of symptoms which are caused by disturbances in vessel wall stability. Those active ingredients include inter alia natural substances from the group of polyphenols (for example flavonoids) and triterpenes (saponins). They are administered in the form of pure substances, substance mixtures or vegetable extracts in the form of teas, tablets, creams or gels.

The effective flavonoids include rutin, a compound which is known inter alia from buckwheat. Besides rutin (also referred to as rutosid) derivatives such as for example troxerutin (trihydroxyethylrutin) are also used, as well as further partially synthetically produced hydroxyglycosides. The flavonoid rutin is to be found inter alia in the leaves of buckwheat (Fagopyrum esculentum). Further active ingredients which fall into the group of flavonoids are the anthocyanin and flavonoid mixtures of preparations which contain defined extracts of red vine leaves, and diosmin which is obtained inter alia from the peel of citrus fruits.

The saponins which have a vasoprotective effect include ruscogenins from butcher's broom (Ruscus aculeatus) and the saponin mixture aescin from the seeds of the horse chestnut (Aesculum hippocastanum).

The principle of the preparations, known from the state of the art, for the treatment of vessel wall insufficiency is in particular that of only increasing the stability of the vessel walls with the above-mentioned active ingredients and thus reducing their permeability in relation to solid and liquid blood components. Thus the membrane-stabilising action of flavonoids of the rutin type is presumably based on inhibition of hyaluronidase. Hyaluronic acid is one of the membrane-stabilising components of the connective tissues layer. Hyaluronidase is a body-specific enzyme which catalyses the breakdown of hyaluronic acid in the connective tissue and inhibition of that enzyme leads to a shift in the enzymatic equilibrium with the consequence that the body-specific processes which enhance membrane stability dominate the equilibrium. The ruscogenins have a toning effect on the veins while it is rather dilation that is promoted in the case of the arteries. In addition ruscogenins in vitro_markedly inhibit the enzyme elastase. Elastase is made responsible for hydrolytic cleaving of the extracellular matrix and the endothelium cell membranes at the vessels. The saponin mixture aescin also inhibits the elastase and additionally the collagenase which catalyses the breakdown of the connective tissue substance collagen. Aescin has significant vasoprotective effects (strengthening weak veins) and vein-toning effects (preventing vessel leakages). In clinical studies with patients suffering from chronic venous insufficiency (CVI), it was found that aescin improves the stability of the capillaries.

One problem is that the above-mentioned active ingredients are very polar in their original form and, in the specified forms of application, it is doubtful whether the active ingredient in all cases actually reaches the desired location of action in order there to deploy its effect. A further problem with the preparations available at the present time is that the complex of symptoms, vessel wall insufficiency, which involves inter alia hematomas or edemas, is generally only treated with active ingredients which have vasoprotective or vein-toning properties respectively.

### BRIEF DESCRIPTION OF THE INVENTION

There is therefore a need for a preparation whose effects cover as much as possible all the complex of symptoms involved with vessel wall insufficiency. In this respect the preparation should guarantee that the active substances actually reach the desired location of action in order there to deploy their effect.

In accordance with the present invention that object is attained by a combined cosmetic or therapeutic preparation having a carrier system comprising membrane-forming lipids and at least two active ingredients which are selected from at least two of the groups (a) anti-coagulants, (b) vasoprotective agents and (c) microcirculation-promoting substances.

Anti-coagulants are substances which inhibit blood coagulation. In order to speed up for example the dissolution of a blood clot or a hematoma, it is advantageous to use anti-coagulants. The function of the vasoprotective agents is in particular a prophylactic one and causes stabilisation of the vessel wall, the consequence of which is an improvement in vessel wall density and a reduction in permeability in relation to blood components. Microcirculation-promoting substances stimulate the circulation of blood in the capillary region of what is referred to as the end flow path. That effect of promoting blood circulation is particularly advantageous for the processes which occur upon breakdown of hematomas and edemas.

The combination of the specified groups of active ingredients with each other in accordance with the invention provides a large number of advantageous combinations of active ingredients for cosmetic or prophylactic or therapeutic application in relation to complexes of symptoms which involve the formation of edemas or hematomas such as for example vessel wall insufficiency.

In accordance with the present invention those active ingredients are combined with a carrier system comprising membrane-forming lipids. That carrier system serves essentially as a transport system for the specified combinations of active ingredients. That transport system according to the invention ensures that, upon use thereof, the active ingredients actually arrive at the desired location of action thereof in order there to deploy their effect. In accordance with the invention there is therefore provided a combined cosmetic or therapeutic preparation having a carrier system comprising membrane-forming lipids and at least two active ingredients which are selected from at least two of the groups

(a) anti-coagulants,

(b) vasoprotective agents and

(c) microcirculation-promoting substances.

The invention also includes the method for making the preparation from its ingredients.

### DETAILED DESCRIPTION OF THE INVENTION

One of the advantageous combinations of active ingredients in accordance with the invention is distinguished in that the active ingredients are selected from the groups anti-coagulants (a) and vasoprotective agents (b). The combination of vasoprotective agents and anti-coagulants is advantageous for the reason that the stability of the vessel walls is increased by the vasoprotective agents prophylactically and possibly also therapeutically while the anti-coagulants locally prevent the formation of hematomas and clots and promote the breakdown of clots and hematomas which are possibly already present.

A further preferred combination of the active ingredients provides that the active ingredients are selected from the groups anti-coagulants (a) and microcirculation-promoting substances (c). In that respect the blood clot-inhibiting action of the anti-coagulants is advantageously promoted by the circulation-promoting action of the microcirculation-promoting substances.

In a further embodiment of the present invention the active ingredients are selected from the groups vasoprotective agents (b) and microcirculation-promoting substances (c). That combination makes it possible to increase the microcirculation and at the same time, by means of the vessel wall stabilising action of the vasoprotective agents, to guarantee that circulation promotion does not involve increased transudation.

A particularly preferred embodiment of the present invention is characterised in that the active ingredients are selected from the groups anti-coagulants (a), vasoprotective agents (b) and microcirculation-promoting substances (c). The triple combination of those active ingredients represents an optimum combination of active ingredients for cosmetic or prophylactic or therapeutic treatment of complexes of symptoms which involve the formation of edemas or hematomas, such as for example vessel wall insufficiency.

The carrier system of the combined preparation is preferably vesicular. In accordance with the present invention the term vesicular carrier system comprising membrane-forming lipids is used to denote double-layer membrane vesicles or single-layer nanoparticles. So-called liposomes are also counted amongst the double-layer and also multi-layer vesicles. In that respect the active ingredients can be present both in the interior of the vesicles in a solution and also incorporated into or between the layers. In addition in accordance with the invention the carrier system can also function in the non-vesicular state, for example as an aggregate of a plurality of layers, as a carrier system for the active ingredients.

The membrane-forming lipids of the carrier system of the combined preparation according to the invention preferably include the membrane-forming lipids from the groups of phospholipids, ceramides and diacylglycosides. If it seems appropriate membrane-forming lipids from different groups can be combined with each other in the form of mixtures.

When using mixtures of various substances from the groups of the membrane-forming lipids, it is preferable if the membrane-forming lipids contain at least 70% by weight of phosphatidylcholine. It is particularly preferred if the membrane-forming lipids contain about 80 to 90% by weight of phosphatidylcholine. The proportion of phosphatidylcholine in the membrane-forming lipids has a decisive influence on the transport properties and the stability of the carrier system. Phosphatidylcholine contents of below about 70% by weight provide a carrier system which has inadequate vesicle stability in vesicular form. Depending on the respective active ingredient or ingredients to be transported and depending on the respective location of action required, at which the active ingredients are to be liberated in order there to deploy their effect, the % proportion by weight of phosphatidylcholine can be varied. With a phosphatidylcholine proportion of about 80% by weight the carrier system penetrates the skin with the active ingredients and there liberates with an in-depth action both hydrophilic and also lipophilic active ingredients. Upon an increase in the phosphatidylcholine content in the carrier system above 80% the in-depth action decreases stepwise. That may be desired for the situation where an action on the part of the active ingredients at a pronounced depth is not required, but rather the active ingredients are to deploy their action in the further upwardly disposed layers.

The anti-coagulants considered for the combined preparation according to the invention include heparins, fucoidans, hirudins, coumarins and mixtures thereof. A distinction is drawn between direct anti-coagulants which interact directly with the clotting factors and indirect anti-coagulants which prevent the synthesis of clotting factors. All those substances prevent the formation of blood clots and thus facilitate blood circulation in particular in the capillary region. For dermal application, use is made in particular of the directly acting macromolecules such as heparins, fucoidans and hirudins as well as synthetically produced low-molecular pentapeptides. An example of an indirect anti-coagulant is acetyl salicylic acid.

In accordance with the invention the term heparins is used to denote both high-molecular and also low-molecular heparins as well as compounds having a similar effect which inhibit for example antithrombin III or the blood coagulation factor Xa. The fucoidans according to the invention also include the high-molecular and low-molecular fucoidans. In accordance with the invention the term hirudin is used to denote hirudins from leech extracts as well as the raw extracts or also purified extracts from leeches, smaller hirudins and gene-technologically produced, recombinant (r-)hirudins as well as other substances which block the active centre of thrombin. The term coumarin in accordance with the invention embraces anti-coagulants of coumarin type, blood coagulation inhibitors derived from coumarin and other active ingredients whose action is based on structural similarity to vitamin K (competitive inhibition).

In a preferred embodiment of the invention fucoidan is contained therein as the anti-coagulant. In a particularly preferred feature there is fucoidan content in an amount of 0.1 to 10% by weight. Satisfactory effectiveness is not established below about 0.1% by weight while solubility is the limiting factor above 10% by weight.

In a further preferred embodiment low-molecular fucoidan (LMD) is contained therein as the anti-coagulant. Particularly preferred in that respect is an amount of 0.1 to 10% by weight.

A combined preparation according to the invention preferably contains vasoprotective agents which include aescin, rutin, diosmin, ruscogenin and mixtures thereof. In accordance with the invention aescin is used to denote saponins and saponin mixtures of aescin type. In addition the term also embraces horse chestnut seed dry extracts which are standardised to aescin. Rutin is used to denote both rutin itself and also further rutosids, oxyrutins, such as for example troxerutin as well as further hydroxyethylrutosids and partially synthetically produced hydroxyglycosides of rutin. The term ruscogenin includes substances from the group of ruscogenin-saponins and extracts from butcher's broom standardised to ruscogenins. In addition defined extracts of red vine leaves can also be considered as vasoprotective agents.

In a preferred embodiment the combined preparation according to the invention contains aescin as the vasoprotective agent. An aescin content of 0.1-7% by weight is particularly preferred. A sufficient vasoprotective effect cannot be established in the range of less than 0.1% by weight while solubility problems occur above 7% by weight.

In one of the preferred combined preparations according to the invention the microcirculation-promoting substances include caffeine, naftidrofuryl, pentoxyfyllin, buflomedil and ginkgo active ingredients and mixtures thereof. In this connection the term ginkgo active ingredients is used to denote standardised extracts of ginkgo and microcirculation-promoting fractions obtained therefrom or pure substances.

In a preferred embodiment of the combined preparation according to the invention caffeine is included as the microcirculation-promoting substance. Particularly preferred in that respect is a content of 0.1 to 2% by weight. No advantageous effect occurs below about 0.1% by weight while solubility problems occur above 2% by weight.

A further particularly preferred embodiment of the combined preparation according to the invention contains aescin, preferably in an amount of 4.0 to 6.0% by weight, particularly preferably 5.0% by weight, low-molecular fucoidan, preferably in an amount of 1.0 to 3.0% by weight, particularly preferably 2.0% by weight, and caffeine, preferably in an amount of 0.5 to 1.5% by weight, particularly preferably 1.0% by weight. The combination of those three active ingredients from a total of three different active ingredient groups, in the specified contents in conjunction with the carrier system according to the invention, affords a combined preparation which is optimised for cosmetic or prophylactic or therapeutic use for the treatment of complexes of symptoms which involve the formation of edemas or hematomas such as for example vessel wall insufficiency.

A preferred embodiment of the combined preparation according to the invention is characterised in that the carrier system contains linoleic acid in stabilised form, preferably in an amount of 2.5 to 4.5% by weight. The term linoleic acid in stabilised form is used in this connection to denote that the linoleic acid, as a component of the carrier system, is stabilised in the carrier system. In other words stabilised linoleic acid is present here bound in the form of the fatty acid constituent linoleic acid of the membrane lipids. That prevents the linoleic acid being chemically modified by body-specific processes and thus losing its effect. Linoleic acid is one of the essential fatty acids brought together under the term Vitamin F. They are inter alia a component of the membrane building blocks of the human skin and the supply of additional linoleic acid slows down the ageing process (for example wrinkle formation) of the human skin.

In a further preferred embodiment the combined preparation according to the invention, besides the stated active ingredients, further contains at least one thermoreceptor-agonist which is selected from the group which includes natural or synthetic capsaicin, preferably in an amount of 0.1 to 1% by weight, particularly preferably in an amount of 0.2 to 0.6% by weight, and nicotinic acid, nicotinic acid amide, nicotinic acid ester or mixtures thereof, preferably in an amount of 0.5 to 5% by weight, particularly preferably in an amount of 0.5 to 3% by weight. The function of the thermoreceptor-agonist in the combined preparation is, in use, by way of the stimulus of the thermoreceptors, to achieve a circulation-promoting effect for the part of the body being treated. In addition to the effect of a contained microcirculation-promoting agent, that also promotes circulation of blood in the larger blood vessels. For the use of a combined preparation according to the invention on the eye, the addition of a thermoreceptor-agonist is possibly to be refrained from, by virtue of the high level of sensitivity of the eye. The combined preparation according to the invention with thermoreceptor-agonist is advantageously suitable however for example for the treatment of vessel insufficiency in hardening of the arteries in the leg as caused by smoking.

In order to conserve the combined preparation according to the invention, a further preferred embodiment of the present invention contains 10-25% by weight of ethanol.

To produce a combined preparation according to the present invention firstly the water-soluble active ingredients from the above-specified groups of anti-coagulants, vasoprotective agents, microcirculation-promoting substances and/or thermoreceptor-agonists in suitable amounts are dissolved to a clear state with agitation in water at a maximum of 40° C. In a further preparation step the fat-soluble active ingredients from the above-specified groups of anti-coagulants, vasoprotective agents, microcirculation-promoting substances and/or thermoreceptor-agonists in suitable amounts are dissolved to a clear state with agitation in an ethanolic lecithin solution at a maximum of 50° C. The two prepared solutions are slowly brought together with turraxing (=homogenisation using a Turrax homogeniser) and then brought to a vesicle diameter size of a maximum of 500 nm by high-pressure homogenisation, extrusion and/or other mechanical size reduction. Aqueous phosphate buffer is then added with steady homogenisation and homogenisation is further continued until a slightly viscous homogenous emulsion is produced. If necessary the pH-value of the emulsion is adjusted with conventional means to about pH 6.5 to 7.5.

A combined preparation according to the invention is preferably incorporated into a cosmetic or pharmaceutical carrier matrix, particularly preferably in a concentration of use of 1.0 to 5.0% by weight. The carrier matrix can involve gel formulations, cream formulations (O/W and W/O emulsions), lotions, mask applications and so forth.

A method of formulating a combined preparation according to the present invention in the form of a gel is to be described in the following terms: a thickener, preferably in an amount of 0.1 to 3.0% by weight, and a non-ionic emulsifier, preferably in an amount of 1.0 to 15.0% by weight, and in a particularly preferred embodiment a co-emulsifier, are completely dissolved in water with slight agitation. At a maximum of 30° C. one of the above-described embodiments of the combined preparation according to the invention, preferably in an amount of 1.0 to 5.0% by weight, is homogenously stirred into that matrix. Then a preserving agent, preferably in an amount of 0.1 to 0.5% by weight, is added and further homogenously stirred in. The gel exhibits a clear to cloudy appearance. The viscosity varies in dependence on the nature and concentration of use of the thickener employed. The pH-value of the gel is adjusted if necessary to about 5.5 to 6.5 with conventional means.

Preferably membrane-forming lipids and at least two active ingredients which are selected from at least two of the groups (a) anti-coagulants, (b) vasoprotective agents and (c) microcirculation-promoting substances, and in a particularly preferred embodiment also at least one thermoreceptor-agonist, are used for the production of a cosmetic or a drug for the prophylaxis and/or treatment of hematomas, preferably hematomas of the lower eyelid, and/or vein complaints.

Further advantages, features and possible uses of the present invention will be clearly apparent from the following Examples and the accompanying Figure.

### EXAMPLE 1

A combined preparation according to the invention is produced in accordance with the above-specified method and includes the following constituents:

| | | |
|---|---|---|
| 16.0% | by weight | ethanol, non-denatured |
| | | (Bundesmonopolverwaltung fur Branntwein |
| | | (Federal Monopoly Administration for Spirits), |
| | | DE) |
| 10.0% | by weight | phospholipids (lecithin/PL 80) |
| 5.0% | by weight | aescin (Synopharm GmbH, D-22885 Barsbüttel) |
| 2.0% | by weight | fucoidan (algae extract, high-purity, Kraeber |
| | | GmbH & Co. D-25474 Ellerbek) |
| 1.0% | by weight | caffeine |
| 0.5% | by weight | potassium dihydrogen phosphate |
| ad 100% | by weight | water |

Firstly fucoidan and caffeine were completely dissolved in water at 40° C., giving a clear, weakly yellowish solution. At the same time the aescin was completely dissolved in a clear, brown ethanolic lecithin solution at a temperature of a maximum of 50° C. The buffer was produced by potassium dihydrogen phosphate being completely dissolved in water with agitation. The pH-value of that solution was adjusted to 11.0 to 12.0 with NaOH solution. Now, with turraxing (=homogenisation using a Turrax homogeniser at 10,000 rpm) the ethanolic lecithin/aescin solution was slowly added to the aqueous fucoidan/caffeine solution and then extruded through a 200 nm polycarbonate filter. Finally with steady homogenisation the phosphate buffer was added and homogenisation was continued until a beige, low-viscosity homogenous emulsion was produced. The pH-value of the emulsion was 6.7. The vesicle size expressed as the diameter of the liposome hollow balls, was determined with a Zetamaster S from Malvern Instruments, UK, using the method of photon correlation spectroscopy (PCS) at 152 nm. If the desired pH-value were not attained directly, if necessary it would be possible to set it to a pH-value of 6.5 to 7.5 with NaOH solution.

### EXAMPLE 2

The combined preparation according to the invention as set forth in Example 1 was incorporated into a gel formulation in a concentration of use of 5.0% by weight. For example a concentration of 1.0 to 5.0% by weight is suitable according to the invention. The gel is only one example for a cosmetic or pharmaceutical carrier matrix which is suitable according to the invention.

The formulation in accordance with Example 2 includes the following constituents:

| | | |
|---|---|---|
| 1.5% | by weight | thickener (Acritamer ®; R.I.T.A., USA) |
| 4.4% | by weight | NaOH solution 10% |
| 5.0% | by weight | emulsifier (Ritabete ®, R.I.T.A., USA) |
| 5.0% | by weight | combined preparation according to the invention |
| | | of Example 1 |
| 0.2% | by weight | preserving agent (Euxyl K 400 ®, Schülke & |
| | | Mayr, DE) |
| ad 100% | by weight | water |

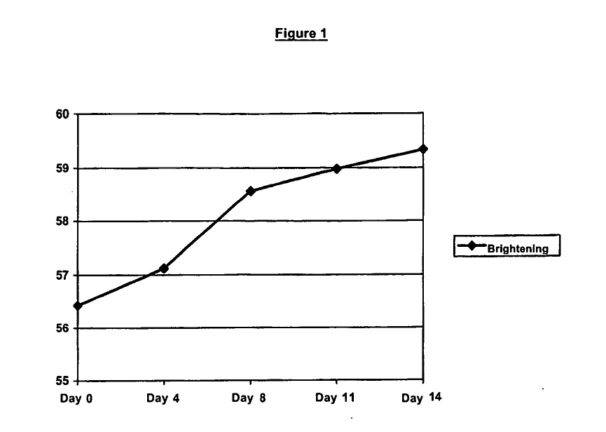

Firstly the thickener was completely dissolved in water, with agitation at ambient temperature, to afford a cloudy, highly viscous gel. The pH-value of that gel was then raised to about 3.3 to 5.8 with 10% NaOH solution. That resulted in a clear gel which is firm to cut. Now the emulsifier, the combined preparation according to the invention and the preserving agent were successively stirred into the gel matrix at a maximum of 30° C. and agitation continued for a further 20 minutes. The resulting cloudy, slightly yellowish gel was of a consistency such that it was firm to cut, and exhibited a pH-value of 5.8. If the desired pH-value were not attained directly, if necessary it could be set to a value of 5.6 to 6.0 by the addition of NaOH solution. The result of using a composition of the combined preparation according to the invention as set forth in Example 2 is shown in FIG. 1.

FIG. 1 shows the result of using a composition of the combined preparation according to the invention as set forth in Example 2.

A combined preparation produced in accordance with Example 2 was applied to 8 experimentees with hematomas in the lower eyelid (“rings under the eyes”) once daily in an amount of 0.1 gram in each case. The colouration of the treated part of the skin was measured prior to and during the treatment with a Chromameter CR-300 (Minolta, Japan). The average values of the colourations of all experimentees in relation to the treatment time in days is shown in FIG. 1. The L* value represents in the colour space in accordance with the L*a*b* colour system the z-co-ordinate and reflects the brightness value of the surface to be measured. In that respect black is to be associated with an L* value of 0 and white an L* value of 100. The lightening effect as a consequence of the positive action of the combined preparation according to the invention on a hematoma of the lower eyelid is clear from FIG. 1. A considerable lightening of the lower eyelid can be detected over a treatment period of 14 days, wherein the lightening effect is at its greatest within 8 days from the beginning of treatment and then progresses more slowly.

What is claimed is:

1. A combined cosmetic or therapeutic preparation having a carrier system comprising membrane-forming lipids and at least two active ingredients which are selected from at least two of the groups
(a) anti-coagulants,
(b) vasoprotective agents and
(c) microcirculation-promoting substances.

2. A combined preparation according to claim 1 wherein the active ingredients are selected from the groups anti-coagulants (a) and vasoprotective agents (b).

3. A combined preparation according to claim 1 wherein the active ingredients are selected from the groups anti-coagulants (a) and microcirculation-promoting substances (c).

4. A combined preparation according to claim 1 wherein the active ingredients are selected from the groups vasoprotective agents (b) and microcirculation-promoting substances (c).

5. A combined preparation according to claim 1 wherein the active ingredients are selected from the groups anti-coagulants (a), vasoprotective agents (b) and microcirculation-promoting substances (c).

6. A combined preparation according to claim 1 wherein the carrier system is vesicular.

7. A combined preparation according to claim 1 wherein the membrane-forming lipids include the groups of phospholipids, ceramides and diacylglycosides.

8. A combined preparation according to claim 1 wherein the membrane-forming lipids contain at least 70% by weight of phosphatidylcholine, preferably about 80-90% by weight of phosphatidylcholine.

9. A combined preparation according to claim 1 wherein the anti-coagulants are selected from heparins, fucoidans, hirudins, pentapeptides, coumarin derivatives and mixtures thereof.

10. A combined preparation according to claim 1 wherein as the anti-coagulant it contains fucoidan, preferably low-molecular fucoidan, particularly preferably in an amount of 0.1-10% by weight.

11. A combined preparation according to claim 1 wherein the vasoprotective agents are selected from aescin, rutin, diosmin, ruscogenin and mixtures thereof.

12. A combined preparation according to claim 1 wherein it contains aescin as the vasoprotective agent, preferably in an amount of 0.1-7% by weight.

13. A combined preparation according to claim 1 wherein the microcirculation-promoting substances are selected from caffeine, naftidrofuryl, pentoxyfyllin, buflomedil and ginkgo active ingredients and mixtures thereof.

14. A combined preparation according to claim 1 wherein it contains caffeine as the microcirculation-promoting substance, preferably in an amount of 0.1-2% by weight.

15. A combined preparation according to claim 1 wherein it contains aescin, preferably in an amount of 4.0 to 6.0% by weight, particularly preferably 5.0% by weight, low-molecular fucoidan, preferably in an amount of 1.0 to 3.0% by weight, particularly preferably 2.0% by weight, and caffeine, preferably in an amount of 0.5 to 1.5% by weight, particularly preferably 1.0% by weight.

16. A combined preparation according to claim 1 wherein the carrier system additionally contains linoleic acid in stabilised form, preferably in an amount of 2.5 to 4.5% by weight.

17. A combined preparation according to claim 1 wherein it further contains at least one thermoreceptor-agonist which is selected from the group which includes natural or synthetic capsaicin, preferably in an amount of 0.1 to 1% by weight, particularly preferably in an amount of 0.2 to 0.6% by weight, and nicotinic acid, nicotinic acid amide, nicotinic acid ester or mixtures thereof, preferably in an amount of 0.5 to 5% by weight, particularly preferably in an amount of 0.5 to 3% by weight.

18. A combined preparation according to claim 1 wherein it further contains 10-25% by weight of ethanol.

19. A method for the production of a preparation for prophylaxis and/or treatment of hematomas comprising combining membrane-forming lipids and at least two active substances which are selected from at least two of the groups (a) anti-coagulants, (b) vasoprotective agents and (c) microcirculation-promoting substances.

20. The method of claim 19 wherein the preparation is for phophylaxis or treatment of hematomas of the lower eyelid, and/or veins.
